SEQUENCE LISTING (15 sequences)

SEQ ID NO: 1 - DNA, 342 bp, Psammomys obesus
  CDS (29)...(247)
gttccaggagattacagctccagccacaatgattgaggtggtttgcaacgaccgtctaggaaagaaagtccgcgttaagtgcaacaccgatgacaccatcggggacttgaagaaactgatagcggcccaaactggcactcgttggaataagatcgttcttaaaaagtggtacacgatttttaaggaccatgtatctctgggagattatgaaatccacgatgggatgaacctggagctttattaccagtagaggggaattcctccaccttgcccaaccttgctttcctctcccatggctcatttaacactgttgtagatgctcatttttttgttaagtgtact

SEQ ID NO: 2 - protein, 73 aa, Psammomys obesus
MIEVVCNDRLGKKVRVKCNTDDTIGDLKKLIAAQTGTRWNKIVLKKWYTIFKDHVSLGDYEIHDGMNLELYYQ

SEQ ID NO: 3 - DNA, 363 bp, Psammomys obesus
tcatgtgaattgttaaaaatgagcatctacaacagtgttaaatgagccatgggagaggaaagcaaggttgggcaaggtggaggaattcccctctactggtaataaagctccaggttcatcccatcgtggatttcataatctcccagagatacatggtccttaaaaatcgtgtaccactttttaagaacgatcttattccaacgagtgccagtttgggccgctatcagtttcttcaagtccccgatggtgtcatcggtgttgcacttaacgcggactttctttcctagacggtcgttgcaaaccacctcaatcattgtggctggagctgtaatctcctggaacctaccgacgacacaacttgta

SEQ ID NO: 4 - DNA, 2180 bp, Homo sapiens
  misc_feature 1594: n = A,T,C or G
accaatctgaagtgggagcgaccggggttcagcgctcgggtgaggagctggtggcgtcggcaggttcgaggcgattcgaggtgagggggtcaagcggagaggctcggagtcggagaaagctgtcgcgacccagccacccagggtctggggtcggtgggaggtgaggccagggagttttgtggcagggctttcaggcggcgagaaggggcggaggcgcggctccccggggttcgcggccctgggacaggggacttggggcctgggagtattcgagtccggggggttgggggcagaacggaaggctcagtaacctggaattttagggcctgggactgggagacttgtaggctggggcttctgggcctgggagacgcctgaagctctgactttgctgcctcccacccaccccccgctttgtgtagctccagctaggatgatcgaggttgtttgcaacgaccgtctggggaagaaggtccgcgttaaatgcaagtatccactggcagccgagaggcagtggtacccgcaggggtgcttggcgtgaggcaggcaactcaatctgtgttgtcggatggttttagttaaacccgggagagggggcggatggggtcctggcagatggtattctgccctagaatgtcctcttcctcgttctacccgcttccatttgccttaactgctctgcgcccagcacggatgataccatcggggaccttaagaagctgattgcagcccaaactggtacccgttggaacaagattgtcctgaagaagtggtgagtgcagcggtagagccactgggtggactggactaggccggaaggttttggttgggggagcgctgcaggcagccctttgcttaggcttggctacctcatttggcctacagactgaagagcctccttagccttatctctgaaatgtctctttttcttaggtacacgatttttaaggaccacgtgtctctgggggactgtatcctttgtgtgactttttgaggaagcatctacatacccagcctcggttatctgttcaaaactaaagtctgcatgagcttatgcatattaagtgtttaacttaatacgtggcacatggtgagcacttagaaaatgggagctaggtaattatttggttggggaggagagtggttggtgaaatagtttgacttcaagttcaaaatatttattactgacctccacctgagtgctggacagtaggtcagggtggggtgagactcaggggtatctattgtgcaaaaggagcagaaactttttacaatttttattttatttatttatttttttgagacagtctggctctgtcgcccaggctggagtgcagtggcacgatcttgacccactgcaacctccacctcccgggttcaagcaattctgcctcagcttcccaagtagctgggattacaggtgtgtgccaccactccctgctagtttttgtatttttagtagagatgaggttccaccatgtatgccaggctagtctcaaactcctggcctcaagtgatcctcccgcctcggcctcccaaagtgctgggattatagcccgtgagccactgtgcctggcctaaaaagaaaaaaaantttttttttttttttttgagatggagttttgctcttgttgcccaggatggagtgcagtggtacaatcttggctcactgcagcctctacctcccaggtttaagcgattttcctgtctcagccttccaagtagctgggattacaggtgcccaccaccacacccagctaactttttgtatttttagtagagacagggtttcatcgtgttggccaggctggtctcaaactcctgacgtcaagtgagccacctgcttcgtcctctcaaagtgctgggattacaggtgtgagccactgcacccagccagaaattcttttgtaaagttttggttctctccaaataaaaatggcctggtccaaaatgggcttccttagccctgggctgagacctcaggccacctgacccggctgtgaatggatgagaggggtggggacggaagtcagagtcaggattccttaacacctttccttcagatgaaatccacgatgggatgaactggagcttaatttcaatagatgagaatcctcatcttcctgccccgctttcctctcccatcctcatcccccacactgggatagatgcttgtttgt

SEQ ID NO: 5 - protein, 33 aa, Homo sapiens
  VARIANT 15: Xaa = Any Amino Acid
MIEVVCNDRLGKKVXVKCNTDDTIGDLKKLIAA

SEQ ID NO: 6 - DNA, 443 bp, Homo sapiens
attatcacagagacattgaaagcgggtatcgaatccactgcagtaaatcttcagtccgcagcaggagaagcagtcctaaaaggaagcgcaatagacactgttcaagtcatcagtcacgttcgaagagccaccgaaggaaaagatccaggagtatagaggatgatgaggagggtcacctgatctgtcaaagtggagacgttctaagagcaagatatgaaatcgtggacactttgggtgaaggagcctttggcaaagttgtagagtgcattgatcatggcatggatggcatgcatgtagcagtgaaaatcgtaaaaaatgtaggccgttaccgtgaagcagctcgttcagaaatccaagtattagagcacttaaatagtactgatcccaatagtgtcttccgatgtgtccagatgctagaatggtttgatcatcatggtcatgtt

SEQ ID NO: 7 - DNA, 1323 bp, Homo sapiens
ttcaaattgaccacatcaggcagatggcgtatcagatctgccagtcaataaattttttacatcataataaattaacccatacagatctgaagcctgaaaatattttgtttgtgaagtctgactatgtagtcaaatataattctaaaatgaaacgtgatgaacgcacactgaaaaacacagatatcaaagttgttgactttggaagtgcaacgtatgatgatgaacatcacagtactttggtgtctacccggcactacagagctcccgaggtcattttggctttaggttggtctcagccttgtgatgtttggagcataggttgcattcttattgaatattaccttggtttcacagtctttcagactcatgatagtaaagagcacctggcaatgatggaacgaatattaggacccataccacaacacatgattcagaaaacaagaaaacgcaagtattttcaccataaccagctagattgggatgaacacagttctgctggtagatatgttaggagacgctgcaaaccgttgaaggaatttatgctttgtcatgatgaagaacatgagaaactgtttgacctggttcgaagaatgttagaatatgatccaactcaaagaattaccttggatgaagcattgcagcatcctttctttgacttattaaaaaagaaatgaaatgggaatcagtggtcttactatatacttctctagaagagattacttaagactgtgtcagtcaactaaacattctaatatttttgtaaacattaaattattttgtacagttaagtgtaaatattgtatgttttgtatcaatagcataattaacttgttaagcaagtatggtcttgataatgcattagaaaaattaaaattaatttttctttttgaaattaccatttttaaatacctttgaaatatcctttgtgtccagtgataaatgtgattgatcttgccttttgtacatggaggtcacctctgaagtgattttttttgagtaaaaggaaatcttgactactttatattcttaaaggaatattctttatatacttcaaatttagaacttaactttaaaagtttttcttctgtaattgttgaacgggtgattattattaactctagataagcaggtactagaaaccaaaactcagaaaatgtttactgttagaattctattaaattttaagtgttgtattctttttcattgggtgatgtcagggtgataaccagacattcatggaaaggcatgcagtttgtccattgtgacagtttgtttaataaaaccacatacaccctttaaaaaaaaaaaaaaaaaaaaaaaaaa

SEQ ID NO: 8 - DNA, 391 bp, Psammomys obesus
atgttcaacacagcagccatccaaggtcctctaatgtcgaggtcggtgttactaactccaccaaacgttgctggcagatcctttctttcaggcgcaattcacgttgtggctactgtggtagcccctgaacttctttgactatcgccgggtttgaccgtgagcaaccttattctagcaagaatttttcaccatgtgctaaaaattcctggtacatagagaccctctaatactttaggtgctaccctacttggacctcgaaataatggtcatctccccttaaggaggtggaacgggttggaacgaaaggagagggtaccgagtaaattgtgacaacatctacgagtaaaaaaacaattcacatgaataaaaactttgatgctgcaaaaaaaaa

SEQ ID NO: 9 - DNA, 102 bp, Homo sapiens
  misc_feature 44: n = A,T,C or G
atgatcgaggttgtttgcaacgaccgtctggggaaaaaggtccncgttaaatgcaacacggatgataccatcggggaccttaagaagctgattgcagcctaa

SEQ ID NO: 10 - DNA, 370 bp, Psammomys obesus
  CDS (29)...(247)
gttccaggagattacagctccagccacaatgattgaggtggtttgcaacgaccgtctaggaaagaaagtccgcgttaagtgcaacaccgatgacaccatcggggacttgaagaaactgatagcggcccaaactggcactcgttggaataagatcgttcttaaaaagtggtacacgatttttaaggaccatgtatctctgggagattatgaaatccacgatgggatgaacctggagctttattaccagtagaggggaattcctccaccttgcccaaccttgctttcctctcccatggctcatttaacactgttgtagatgctcatttttaacaattcacatgaataaaaactttgatgctgcaaaaaaaaa

SEQ ID NO: 11 - DNA, 443 bp, Mus musculus
attaccatagagacgttgaaagcacttaccggatccattgcagtaaatcctcagtcaggagcaggagaagcagccctaagagaaagcgtaatagaccctgtgcaagtcatcagtcgcattcgaagagccaccgaaggaaaagatccaggagtatagaggatgatgaggagggtcacctgatctgtcaaagtggagacgttctaagagcaagatatgaaatcgtggacactttaggtgaaggagcctttggcaaagttgtagagtgcattgatcacggcatggatggcttacatgtagcagtgaaaattgtaaaaaatgtaggacgttaccgggaggcagctcgttctgaaatccaagtattggagcacttgaacagcactgaccccaacagtgtcttccgatgcgtccagatgctagagtggtttgatcatcatggtcatgtt

SEQ ID NO: 12 - DNA, 1549 bp, Mus musculus
  CDS (79)...(1521)
aaagagacgcagcggctggagaggaacgacggcggtttggcgacatttctgcccaaaaggccgcttgcttttgcggagatgcggcattccaaacgaactcactgtcctgattgggatagtagagaaagctggggccatgaaagctacagtggaagtcacaaacgcaagagaaggtctcacagcagtactcaggagaacaggcactgtaaaccacatcatcagtttaaagactcggattgtcactatttagaagcaagatgcttgaatgagagagattatcgggaccggagatacattgatgaatacagaaatgactactgcgaaggatatgttccaagacattaccatagagacgttgaaagcacttaccggatccattgcagtaaatcctcagtcaggagcaggagaagcagccctaagagaaagcgtaatagaccctgtgcaagtcatcagtcgcattcgaagagccaccgaaggaaaagatccaggagtatagaggatgatgaggagggtcacctgatctgtcaaagtggagacgttctaagagcaagatatgaaatcgtggacactttaggtgaaggagcctttggcaaagttgtagagtgcattgatcacggcatggatggcttacatgtagcagtgaaaattgtaaaaaatgtaggacgttaccgggaggcagctcgttctgaaatccaagtattggagcacttgaacagcactgaccccaacagtgtcttccgatgcgtccagatgctagagtggtttgatcatcatggtcatgtttgtattgtgtttgagctgctgggacttagtacctatgattttattaaagaaaatagttttctgccatttcaaattgatcacatcaggcaaatggcttatcagatctgccagtctataaattttttacatcataataaattaacacacacggacctaaaacctgaaaatattttatttgtgaagtctgactatgtagtcaaatacaattctaaaatgaaacgagatgagcgcacattgaaaaacacagatatcaaagttgttgattttggaagtgcaacatatgacgacgaacatcatagtactttggtgtccacaaggcactacagggctccagaggtcattttggctctaggttggtctcagccttgtgatgtttggagcataggctgcattcttattgagtactaccttgggttcacagtctttcagacccacgatagtaaagagcacctggcaatgatggagcggatcttaggacccatcccagcacatatgatccagaagacaaggaaacgcaagtatttccaccataaccagctagattgggacgagcatagttcagctgggagatatgttaggagacgctgcaagccgttaaaggaatttatgctgtgtcatgacgaagagcatgagaagctgtttgacctggttcgaagaatgttggagtatgacccagcgagaaggatcaccttggatgaagcattgcagcaccctttctttgacttattaaaaaggaaatgagtgggagtcagggcggccgcaccgc

SEQ ID NO: 13 - DNA, 1740 bp, Mus musculus
  CDS (86)...(1534)
atcgtcgtaatcgtttgcagacttctcgccgtcgccttgtaagctttgtcttcgccttgcaagctttgtcttcagggttggaaagatgagacattcaaagagaacttactgtcctgactgggatgaaagagactgggattatggaacatggagaagcagcagcagtcacaaaagaaagaagagatcacatagcagcgcccgtgagcaaaagcgctgcaggtacgatcactccaaaacgacagacagctattatctggaaagcagatccataaatgagaaagcttatcatagtcgacgctatgttgatgaatacaggaatgactacatgggctacgagccagggcatccctatggagaacctggaagcagataccagatgcatagtagcaagtcctctggtaggagtggaagaagcagttacaaaagtaaacacaggagtcgccaccacacatcgcagcaccattcacacgggaagagtcaccgaaggaaaagatcgaggagtgtagaggatgatgaggagggtcacctgatctgtcagagtggagacgtactaagtgcaagatatgaaattgttgatactttaggtgaaggtgctttcggaaaagtggtggaatgcatcgatcataaagtgggaggtagacgtgtagcagtaaaaatagttaaaaatgtggatagatactgtgaagctgctcaatcggaaatacaagttttggaacacttgaatacaacagacccccatagtactttccgttgtgtccagatgttggagtggtttgagcatcgaggtcacatttgcattgtgtttgaacttctggggcttagtacttatgatttcattaaggaaaacagttttctgccgtttcgaatggatcatatcaggaagatggcatatcaaatatgcaaatctgtaaactttttgcatagtaataaattgactcatacagacttgaagcctgaaaacatcttatttgtgaagtctgactacacagaggcttataatcccaaaatgaaacgtgatgaacgtactatagtaaatccagatattaaagtggtggactttggaagtgcaacatatgatgatgaacaccacagcacattggtatctacaagacattatagagcaccggaagttattttagccctcgggtggtcacagccatgtgatgtctggagcataggatgtattcttatcgagtattatcttggatttacagttttttcgactcatgatagcagggaacatttagcaatgatggaaaggattcttggaccactaccaaagcacatgatacagaaaaccaggaaacgcagatatttccatcatgatcgattagattgggatgaacacagttctgctggcagatatgtttctcggcgctgtaaacctctgaaggagtttatgctatctcaggatgccgaacatgagcttctctttgacctcattgggaaaatgttggagtatgatcccgccaaaagaattactctcaaagaagccctaaagcatcctttcttttacccacttaaaaagcatacgtgatttataaacacagtgctctgaaaggaatcttacagactgtatcagtctagcttttaattaagttattttgtatagcttaatttgtaaaacattttatgttttttagatgctttattaaatacatggccaaaccaaataacatctttcagtaattatagaatgatttatttggaataaaatttgtgcttatgaatgtaaaaaaa

SEQ ID NO: 14 - DNA, 1834 bp, Homo sapiens
  CDS (156)...(1607)
atttttagataatcattaaagaccacagaaaatgtaacagatcctactcttcaaaataattgctattcagtattaaaacgagcagtcagctgcgtgattcccgtgattgcgttacaagctttgtctccttcgacttggagtctttgtccaggacgatgagacactcaaagagaacttactgtcctgattgggatgacaaggattgggattatggaaaatggaggagcagcagcagtcataaaagaaggaagagatcacatagcagtgcccaggagaacaagcgctgcaaatacaatcactctaaaatgtgtgatagccattatttggaaagcaggtctataaatgagaaagattatcatagtcgacgctacattgatgagtacagaaatgactacactcaaggatgtgaacctggacatcgccaaagagaccatgaaagccggtatcagaaccatagtagcaagtcttctggtagaagtggaagaagtagttataaaagcaaacacaggattcaccacagtacttcacatcgtcgttcacatgggaagagtcaccgaaggaaaagaaccaggagtgtagaggatgatgaggagggtcacctgatctgtcagagtggagacgtactaagtgcaagatatgaaattgttgatactttaggtgaaggagcttttggaaaagttgtggagtgcatcgatcataaagcgggaggtagacatgtagcagtaaaaatagttaaaaatgtggatagatactgtgaagctgctcgctcagaaatacaagttctggaacatctgaatacaacagaccccaacagtactttccgctgtgtccagatgttggaatggtttgagcatcatggtcacatttgcattgtttttgaactattgggacttagtacttacgacttcattaaagaaaatggttttctaccatttcgactggatcatatcagaaagatggcatatcagatatgcaagtctgtgaattttttgcacagtaataagttgactcacacagacttaaagcctgaaaacatcttatttgtgcagtctgactacacagaggcgtataatcccaaaataaaacgtgatgaacgcaccttaataaatccagatattaaagttgtagactttggtagtgcaacatatgatgacgaacatcacagtacattggtatctacaagacattatagagcacctgaagttattttagccctagggtggtcccaaccatgtgatgtctggagcataggatgcattcttattgaatactatcttgggtttaccgtatttccaacacacgatagtaaggagcatttagcaatgatggaaaggattcttggacctctaccaaaacatatgatacagaaaaccaggaaacgtaaatattttcaccacgatcgattagactgggatgaacacagttctgccggcagatatgtttcaagagcctgtaaacctctgaaggaatttatgctttctcaagatgttgaacatgagcgtctctttgacctcattcagaaaatgttggagtatgatccagccaaaagaattactctcagagaagccttaaagcatcctttctttgaccttctgaagaaaagtatatagatctgtaattggacagctctctcgaagagatcttacagactgtatcagtctaatttttaaattttaagttattttgtacagctttgtaaattcttaacatttttatattgccatgtttattttgtttgggtaatttggttcattaagtacatagctaaggtaatgaacatctttttcagtaattgtaaagtgatttattcagaataaattttttgtgcttatga

SEQ ID NO: 15 - protein, 73 aa, Psammomys obesus
MIEVVCNDRLGKKVHVKCNTDDTIGDLKKLIAAQTGTRWNKIVLKKWYTIFKDHVSLGDYEIHDGMNLELYYQ

(19) United States
(12) Patent Application Publication
(10) Pub. No.: US 2006/0194233 A1
(43) Pub. Date: Aug. 31, 2006

(54) Ligand of the protein "beacon"

(76) Inventors: Greg Collier, Victoria (AU); Ken Walder, Victoria (AU); Paul Zimmet, Victoria (AU)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/354,263

(22) Filed: Feb. 13, 2006

Related U.S. Application Data
(63) Continuation of application No. 09/959,164, filed on Jan. 18, 2002.

(30) Foreign Application Priority Data
Apr. 23, 1999 (AU) PP 9919
Mar. 24, 2000 (AU) PQ 6454

(51) Int. Cl. C12Q 1/68; C07H 21/04; C12P 21/06; C12N 9/12

(52) U.S. Cl. 435/6; 435/69.1; 435/194; 435/320.1; 435/325; 536/23.2

(57) ABSTRACT

The present invention relates generally to a ligand for a protein associated with modulating obesity, diabetes and metabolic energy levels in animals and humans and to genetic sequences encoding the ligand. More particularly, the present invention is directed to a ligand of the protein “beacon” and its homologues. The identification of the ligand molecule permits the development of a range of therapeutic and diagnostic protocols for obesity, diabetes and energy imbalance.

### FIELD OF THE INVENTION

The present invention relates generally to a ligand for a protein associated with modulating obesity, diabetes and metabolic energy levels in animals and humans and to genetic sequences encoding the ligand. More particularly, the present invention is directed to a ligand of the protein “beacon” and its homologues. The identification of the ligand molecule permits the development of a range of therapeutic and diagnostic protocols for obesity, diabetes and energy imbalance.

### BACKGROUND OF THE INVENTION

Bibliographic details of the publications referred to by author in this specification are collected at the end of the description.

The increasing sophistication of recombinant DNA technology is greatly facilitating research and development in the medical, veterinary and allied human and animal health fields. This is particularly the case in the investigation of the genetic bases involved in the etiology of certain disease conditions. One particularly significant condition from the stand point of morbidity and mortality is obesity and its association with non-insulin-dependent diabetes mellitus (NIDDM) and cardiovascular disease.

Obesity is defined as a pathological excess of body fat and is the result of an imbalance between energy intake and energy expenditure for a sustained period of time. Obesity is the most common metabolic disease found in affluent societies. The prevalence of obesity in these nations is alarmingly high, ranging from 10% to upwards of 50% in some subpopulations (Bouchard, 1994). Of particular concern is the fact that the prevalence of obesity appears to be rising consistently in affluent societies and is now increasing rapidly in less prosperous nations as they become more affluent and/or adopt cultural practices from the more affluent countries (Zimmet, 1992).

In Australia, for example, studies using the definition of obesity of BMI>30 have found prevalence rates for obesity of 8.2-9.3% in men and 9.1-11.1% in women (Risk Factor Prevalence Study Management Committee, 1990; Waters and Bennett, 1995). The prevalence rates for obesity are increasing in Australia, as they are in many affluent societies. Bennett and Magnus (1994) found that the mean weight of Australian females aged 20-69 increased by 3.1 kg (from 61.7 to 64.8 kg) from 1980 to 1989, while the corresponding increase in males was 1.8 kg (from 77.0 to 78.8 kg). No change in height was observed during this period. Accordingly, the crude prevalence rates of obesity increased from 8.0 to 13.2% in females and from 9.3 to 11.5% in males (Bennett and Magnus, 1994). All of the above changes were statistically significant (p<0.05).

The high and increasing prevalence of obesity has significant health implications. Obesity has been identified as a key risk indicator of preventable morbidity and mortality due to disease such as NIDDM and cardiovascular disease (National Health and Medical Research Council, 1996). The annual costs of obesity in Australia, for example, associated with these and other disease conditions have been conservatively estimated at AU$810 million (National Health and Medical Research Council, 1996).

A genetic basis for the etiology of obesity is indicated inter alia from studies in twins, adoption studies and population-based analyses which suggest that genetic effects account for 25-80% of the variation in body weight in the general population (Bouchard 1994; Kopelman et al, 1994; Ravussin, 1995). It is considered that genes determine the possible range of body weight in an individual and then the environment influences the point within this range where the individual is located at any given time (Bouchard, 1994).

Obesity is a complex and heterogeneous disorder and of considerable relevance to society. However, despite numerous studies into genes thought to be involved in the pathogenesis of obesity, there have been surprisingly few significant findings in this area. In addition, genome-wide scans in various population groups have not produced definitive evidence of the chromosomal regions having a major effect on obesity.

The hypothalamus has long been recognized as a key brain area in the regulation of energy intake. Early studies led to the dual-centre hypothesis which proposed that two opposing centres in the hypothalamus were responsible for the initiation and termination of eating, the lateral hypothalamus (LHA; “hunger centre”) and ventromedial hypothalamus (VMH; “satiety centre”; Stellar, 1954). The dual-centre hypothesis has been repeatedly modified to accommodate the increasing information about the roles played by various other brain regions, neurotransmitter systems, and hormonal and neural signals originating in the gut on the regulation of food intake. In addition to the LHA and VMH, the paraventricular nucleus (PVN) is now considered to have an important integrative function in the control of energy intake.

A large number of neurotransmitters have been investigated as possible hypothalamic regulators of feeding behaviour including neuropeptide Y (NPY), glucagon-like peptide 1 (GLP-1), melanin-concentrating hormone (MCH), serotonin, cholecystokinin and galanin. Some of these neurotransmitters stimulate food intake, some act in an anorexigenic manner and some have diverse effects on energy intake depending on the site of administration. For example, gamma-aminobutyric acid (GABA) inhibits food intake when injected into the LHA, but stimulates eating when injected into the VMH or PVN (Leibowitz, 1985). Feeding behaviour is thought to be greatly influenced by the interaction of stimulatory and inhibitory signals in the hypothalamus.

In work leading up to the present invention, the inventors made a significant break through in determining a genetic basis of obesity by identifying a genetic sequence referred to as “beacon which is differentially expressed in lean and obese animals. This genetic sequence is associated with energy balance and is also involved in modulating obesity and diabetes. See International Patent Application No. PCT/AU98/00902 filed on 30 Oct., 1998 in the names of International Diabetes Institute and Deakin University. This International application is incorporated herein by reverence. The inventors now propose the presence of a ligand capable of interacting with the protein, beacon. The interaction between beacon and its ligand is proposed to be a factor in obesity, diabetes and energy imbalance. The identification of a beacon-interacting ligand provides the means for developing a range of therapeutic and diagnostic agents for conditions such as obesity and diabetes.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier, i.e. <400>1, <400>2, etc. A sequence listing is provided at the end of the description.

Accordingly, one aspect of the present invention provides a ligand of a protein or a derivative, homologue, analogue or mimetic of said protein which protein is produced in larger amounts in hypothalamus tissue of obese animals compared to lean animals.

Another aspect of the present invention provides a ligand or a derivative, homologue, analogue or mimetic which ligand is capable of interacting with a protein which is produced in a larger amount of hypothalamus tissue of obese animals compared to lean animals and which is encoded by a nucleotide sequence substantially as set forth in <400>1 or <400>4 or a nucleotide sequence having at least about 50% similarity thereto or a nucleotide sequence capable of hybridizing <400>1 or <400>4 under low stringency conditions.

Yet another aspect of the present invention is directed to a ligand capable of interacting with a protein which comprises the amino acid sequence substantially as set forth in <400>2 or <400>5 or an amino acid sequence having at least 50% similarity thereto and wherein said protein is produced in larger amounts in hyperthalamus tissue of obese animals compared to lean animals.

In still another aspect of the present invention, the nucleotide sequence substantially as set forth in <400>6 and/or <400>7 or a nucleotide sequence having at least about 50% similarity to one or both of <400>6 or <400>7 after optimal alignment or a nucleotide sequence capable of hybridizing to one or both of <400>6 or <400>7 under low stringency conditions.

In still yet another aspect of the present invention, the ligand comprises a nucleotide sequence substantially as set forth in FIG. 2 or FIG. 3 or a nucleotide sequence having at least about 50% similarity to a nucleotide sequence in FIG. 2 or FIG. 3 after optimal alignment or a nucleotide sequence capable of hybridizing to a nucleotide sequence in FIG. 2 or FIG. 3 under low stringency conditions.

Another aspect of the present invention contemplates a method of identifying a ligand of the protein beacon or its derivatives, said method comprising introducing a first genetic construct in a yeast strain, said genetic construct comprising a nucleotide sequence encoding all or part of beacon fused to a nucleotide sequence encoding one of a DNA binding (DB) domain or an activation domain (AD) and introducing a second genetic construct into said yeast comprising a cDNA, said second genetic construct comprising elements of a cDNA library fused to a nucleotide sequence encoding the other of a DB domain or AD domain and selecting yeast cells which comprise both genetic constructs and in which a reporter gene has been subjected to two-hybrid dependent transcription.

Yet another aspect of the present invention contemplates a method for modulating expression of beacon ligand in a mammal, said method comprising contacting the beacon ligand gene with an effective amount of a modulator of beacon ligand expression for a time and under conditions sufficient to up-regulate or down-regulate or otherwise modulate expression of beacon ligand.

Still another aspect of the present invention contemplates a method of modulating activity of beacon in a mammal, said method comprising administering to said mammal a modulating effective amount of a soluble beacon ligand or a derivative thereof for a time and under conditions sufficient to increase or decrease beacon activity.

Still yet another aspect of the present invention contemplates, in one embodiment, a composition comprising a soluble form of beacon ligand or a modulator of beacon ligand expression and one or more pharmaceutically acceptable carriers and/or diluents.

### BRIEF DESCRIPTION OF THE FIGURES

FIG. 1A is a representation showing the nucleotide sequence of both strands of a differentially expressed band in hypothalamus tissue of lean and obese Psammomys obesus corresponding to beacon. The amino acids encoded by each codon are shown above in single letter code and the numbering refers to the amino acid position from the start codon.

FIG. 1B is a representation of a nucleotide and corresponding amino acid sequence of the short form of beacon. Note that amino acid 15 may be His or Arg and the corresponding codon may be CGC or CAC, respectively.

FIG. 2 is a representation of the partial nucleotide sequence of pPC86 clone 31 which encodes a beacon ligand.

FIG. 3 is a representation of multiple sequence alignments of pPC86 clone 31, which encodes a beacon ligand, with CLK4M, STYMA and CLK1H.

FIG. 4 is a diagrammatic representation of the yeast two-hybrid screening protocol.

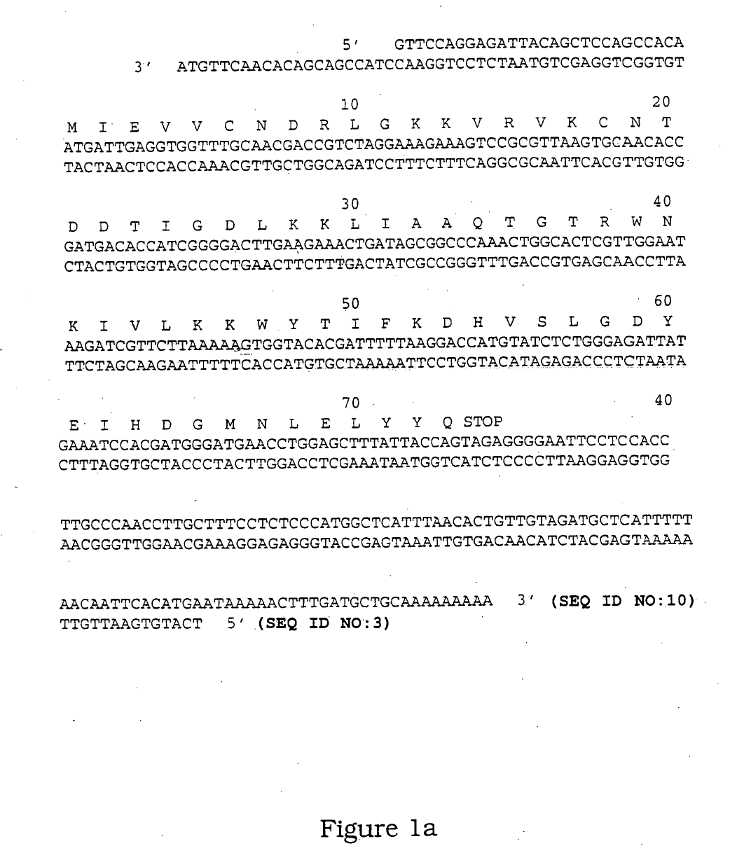

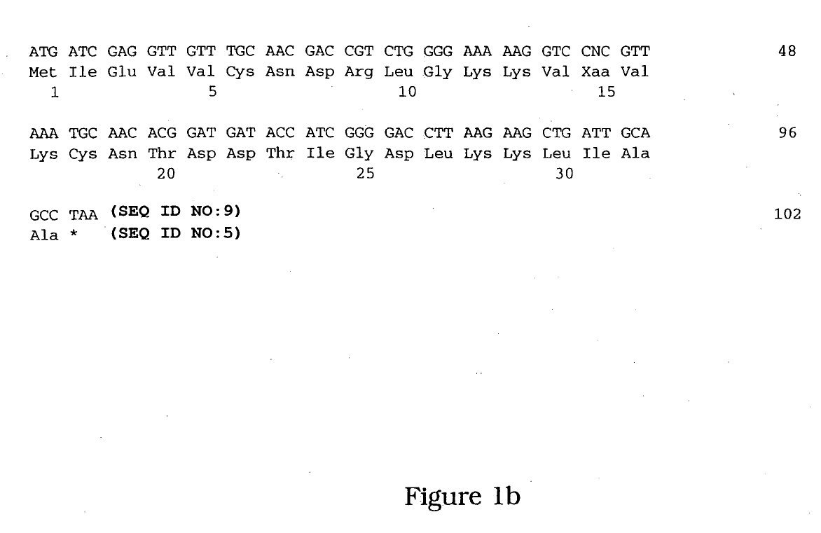

### DETAILED DESCRIPTION OF THE PREFERRED EMBODIMENTS

The present invention is predicated in part on the identification of a ligand for the product of a gene associated inter alia with regulation of energy balance, obesity and diabetes. The preferred gene is referred to as “beacon” and was identified following differential screening of hypothalamic mRNA between lean and obese animals (see International Patent Application No. PCT/AU98/00902).

The term “ligand” means a peptide, polypeptide or protein which binds, forms a close interaction to or which otherwise associates with a protein involved in energy imbalance, obesity and diabetes. Examples of ligands contemplated by the present invention include cell bound receptors, soluble receptors, intracellular ligands, extracellular ligands and partners in a complex comprising the protein involved in energy imbalance, obesity and diabetes. A single ligand may be involved in interaction with the protein or a complex of two or more ligands may be required to from a complex with the subject protein. The term “ligand” also includes binding or interacting partners, cell bound receptors and soluble receptors.

The terms “lean” and “obese” are used in their most general sense but should be considered relative to the standard criteria for determining obesity. Generally, for human subjects the definition of obesity is BMI>30 (Risk Factor Prevalence, 1990; Waters and Bennett, 1995).

Conveniently, an animal model may be employed to study the effects of obese and lean animals. In particular, the present invention is exemplified using the Psammomys obesus (the Israeli sand rat) animal model of dietary-induced obesity and NIDDM. In its natural desert habitat, an active lifestyle and saltbush diet ensure that they remain lean and normoglycemic (Shafrir and Gutman, 1993). However, in a laboratory setting on a diet of ad libitum chow (on which many other animal species remain healthy), a range of pathophysiological responses are seen (Barnett et al., 1994a, b; Barnett et al., 1995). By the age of 16 weeks, more than half of the animals become obese and approximately one-third develop NIDDM. Only hyperphagic animals go on to develop hyperglycemia, highlighting the importance of excessive energy intake in the pathophysiology of obesity and NIDDM in Psammomys obesus (Collier et al., 1997a; Walder el al., 1997a). Other phenotypes found include hyperinsulinemia, dyslipidemia and impaired glucose tolerance (Collier et al., 1997a, b). Psammomys obesus exhibit a range of bodyweight and blood glucose and insulin levels which forms a continuous curve that closely resembles the patterns found in human populations, including the inverted U-shaped relationship between blood glucose and insulin levels known as “Starling's curve of the pancreas” (Barnett et al., 1994a; DeFronzo, 1988). It is the heterogeneity of the phenotypic response of Psammomys obesus which make it an ideal model to study the etiology and pathophysiology of obesity and NIDDM.

A preferred aspect of the present invention is directed to a ligand capable of interacting with “beacon”, the product of the gene “beacon”. The nucleotide sequence of beacon is set forth in <400>1 and <400>4. The amino acid sequence of beacon is set forth in <400>2 and <400>5.

Accordingly, another aspect of the present invention provides a ligand or a derivative, homologue, analogue or mimetic which ligand is capable of interacting with a protein which is produced in a larger amount of hypothalamus tissue of obese animals compared to lean animals and which is encoded by a nucleotide sequence substantially as set forth in <400>1 or <400>4 or a nucleotide sequence having at least about 50% similarity thereto or a nucleotide sequence capable of hybridizing <400>1 or <400>4 under low stringency conditions.

According, another aspect of the present invention is directed to a ligand capable of interacting with a protein which comprises the amino acid sequence substantially as set forth in <400>2 or <400>5 or an amino acid sequence having at least 50% similarity thereto and wherein said protein is produced in larger amounts in hyperthalamus tissue of obese animals compared to lean animals.

Reference herein to similarity is generally at a level of comparison of at least 15 consecutive or substantially consecutive nucleotides or at least 5 consecutive or substantially consecutive amino acid residues. Generally, similarity or identity is determined after optimal alignment of the sequences.

The term “similarity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particularly preferred embodiment, nucleotide and sequence comparisons are made at the level of identity rather than similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as for example disclosed by Altschul et al. (1997). A detailed discussion of sequence analysis can be found in Unit 19.3 of Ausubel et al. (1998).

The terms “sequence similarity” and “sequence identity” as used herein refers to the extent that sequences are identical or functionally or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, lie, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (i.e. the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

Reference herein to a low stringency includes and encompasses from at least about 0 to at least about 15% v/v fornamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C)% (Marmur and Doty, 1962). However, the Tₘ of a duplex DNA decreases by 1° C. with every increase of 1% in the number of mismatch base pairs (Bonner and Laskey, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

The nucleotide sequence or amino acid sequence of the beacon ligand of the present invention may correspond to exactly the same sequence of the naturally occurring ligand or its gene (or corresponding cDNA) or may carry one or more nucleotide or amino acid substitutions, additions and/or deletions.

Any number of approaches may be employed to identify the ligand.

In one particularly useful method, a yeast two-hybrid system is employed. The yeast two-hybrid system is an in vivo genetic technique that can be utilized for the identification of protein:protein interactions. The essence of the two-hybrid system is that interaction between two proteins (X and Y) can be identified by reconstituting active transcription factor dimers. In yeast, these dimers are formed between two fusion proteins, one of which contains a DNA binding (DB) domain fused to the first protein of interest X and the other, an activation domain (AD) fused to a second protein Y. Interaction between DB-X and AD-Y forms a functional transcription factor that activates chromosomally integrated-reporter genes driven by promoters containing the relevant DB binding sites. When a selectable marker such as HIS3 is used as a reporter gene, two-hybrid dependent transcription activation can be monitored by growth on plates lacking histidine. This technique can, therefore, be applied to test whether two known proteins interact or to detect an unknown protein, encoded by a cDNA library, that interacts with a protein of interest.

Accordingly, another aspect of the present invention contemplates a method of identifying a ligand of the protein beacon or its derivatives, said method comprising introducing a first genetic construct in a yeast strain, said genetic construct comprising a nucleotide sequence encoding all or part of beacon fused to a nucleotide sequence encoding one of a DNA binding (DB) domain or an activation domain (AD) and introducing a second genetic construct into said yeast comprising a cDNA, said second genetic construct comprising elements of a cDNA library fused to a nucleotide sequence encoding the other of a DB domain or AD domain and selecting yeast cells which comprise both genetic constructs and in which a reporter gene has been subjected to two-hybrid dependent transcription.

According to this embodiment, if the cDNA from the cDNA library encodes a binding partner for beacon, then a dimer forms and the DB and AD domains permit transcription of the reporter gene.

In one embodiment, the yeast reporter gene is HIS3 although any other reporter gene may be employed. Preferably, the reporter gene provides a selectable marker.

In a particularly preferred embodiment, the ligand comprises the nucleotide sequence substantially as set forth in <400>6 and/or <400>7 or a nucleotide sequence having at least about 50% similarity to one or both of <400>6 or <400>7 after optimal alignment or a nucleotide sequence capable of hybridizing to one or both of <400>6 or <400>7 under low stringency conditions.

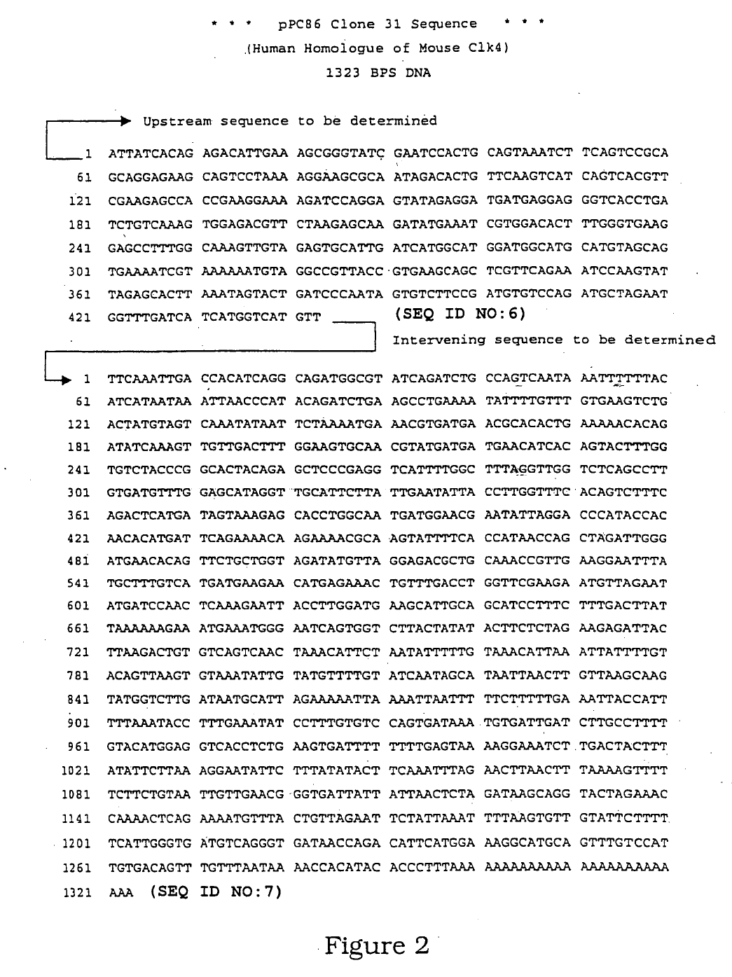

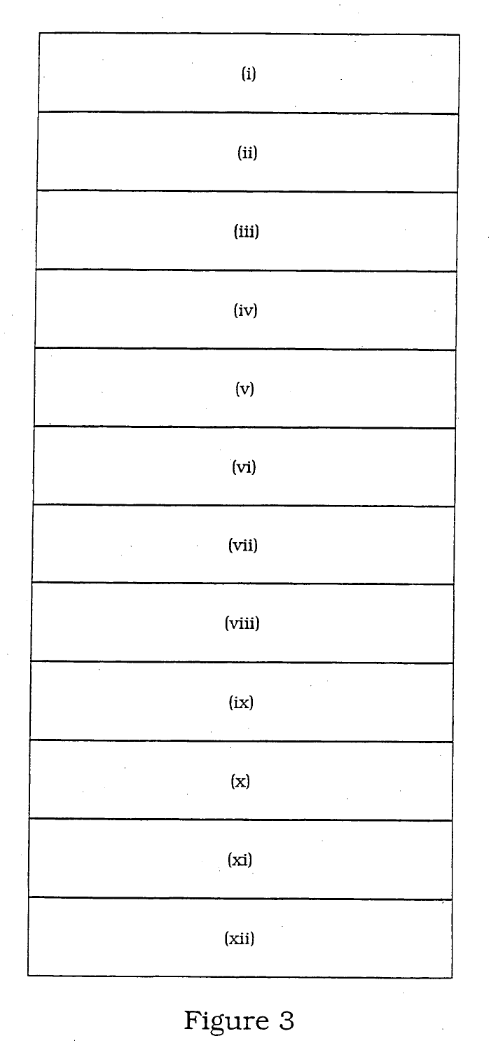

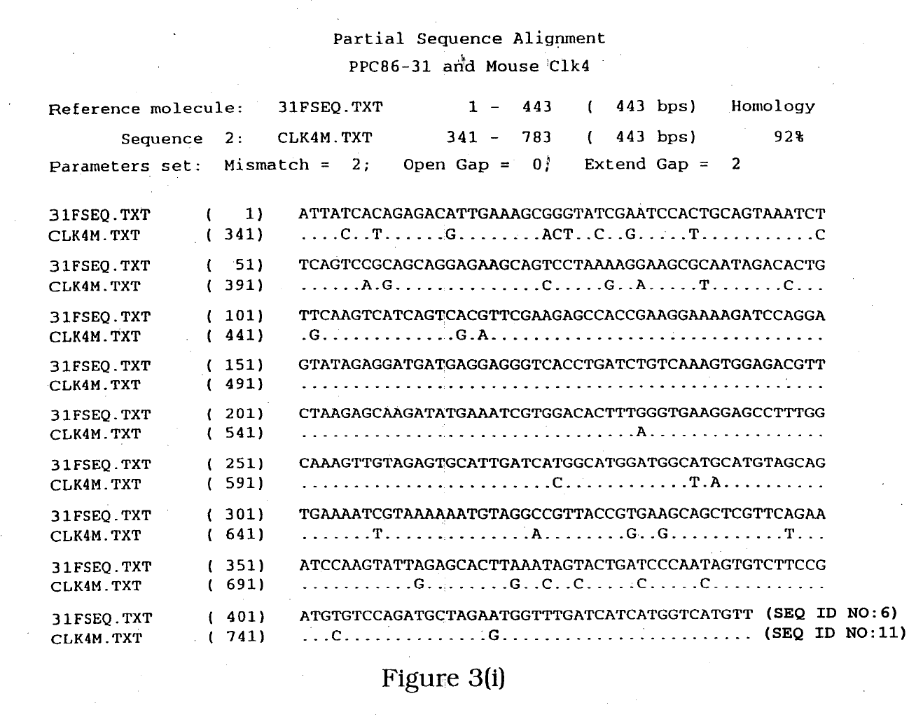

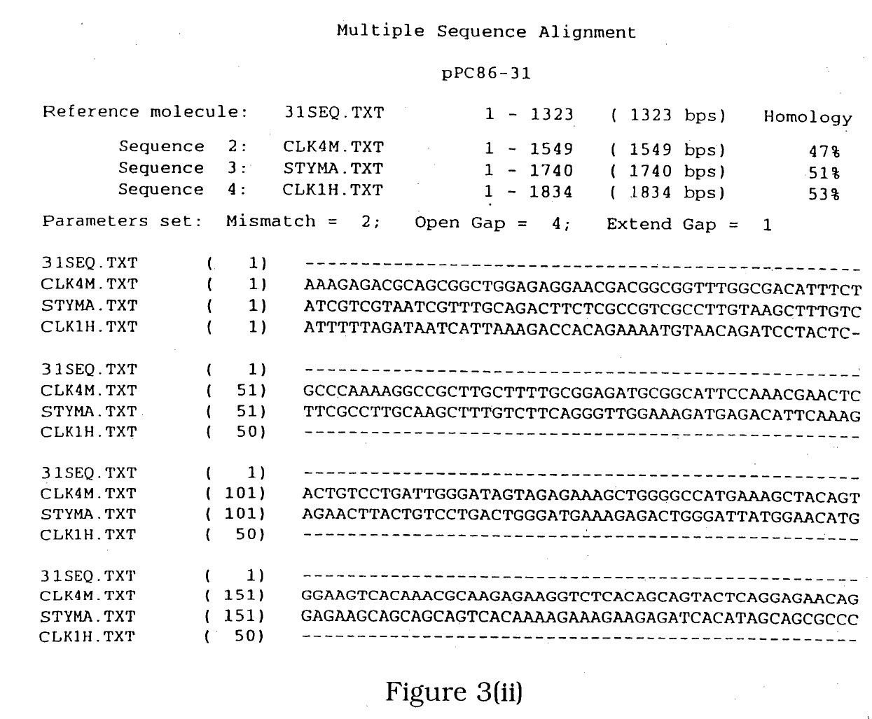

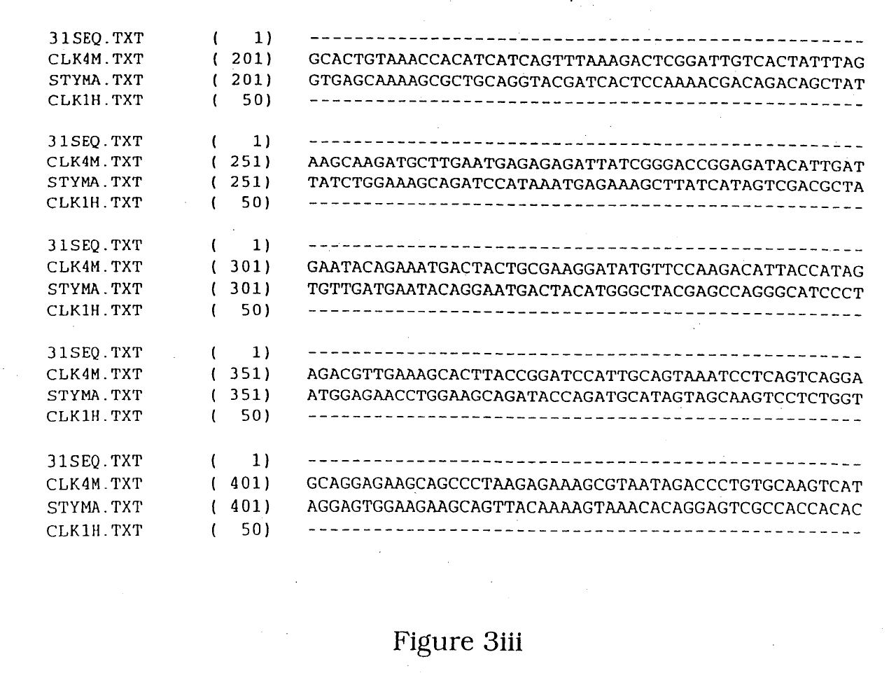

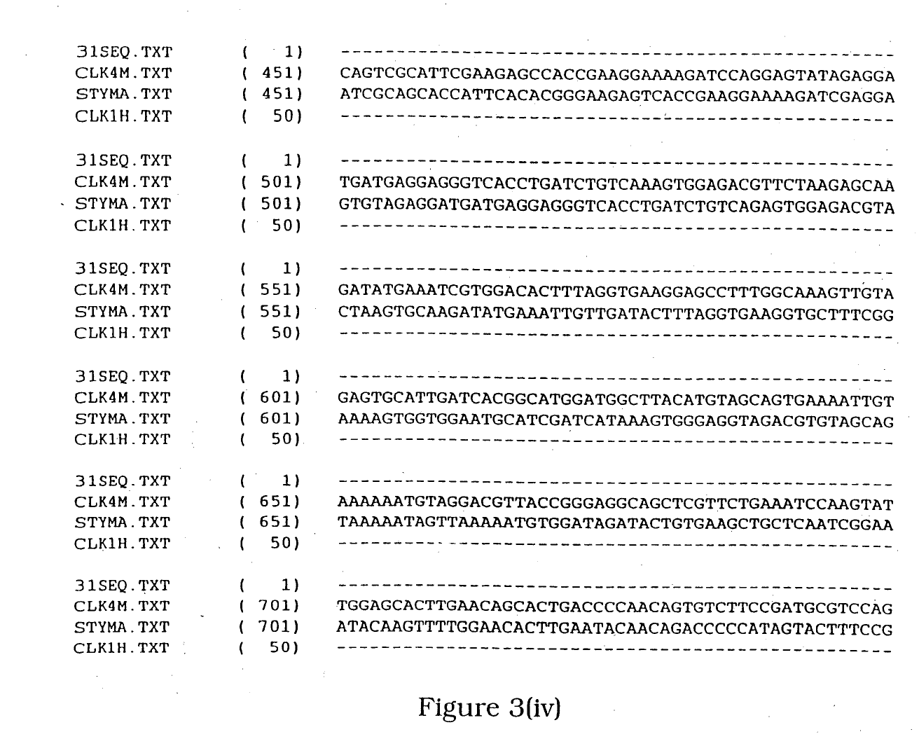

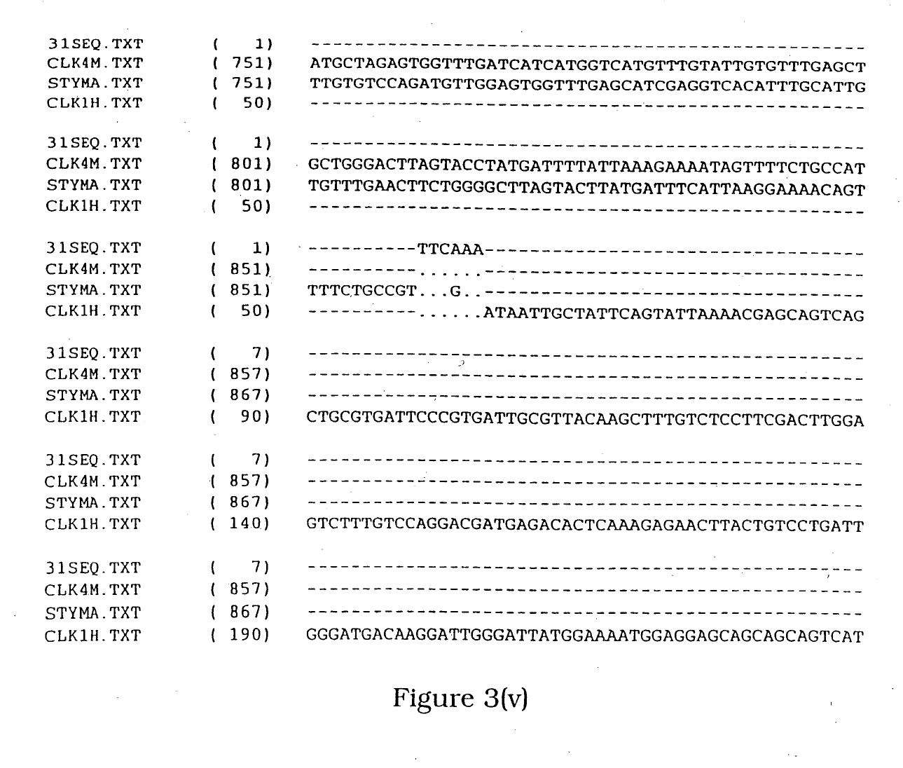

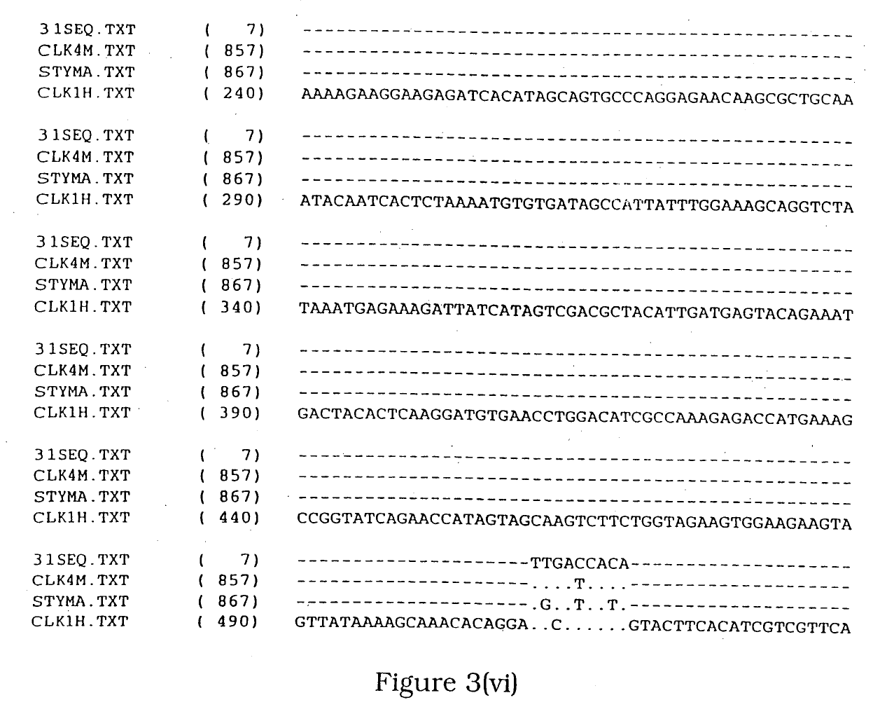

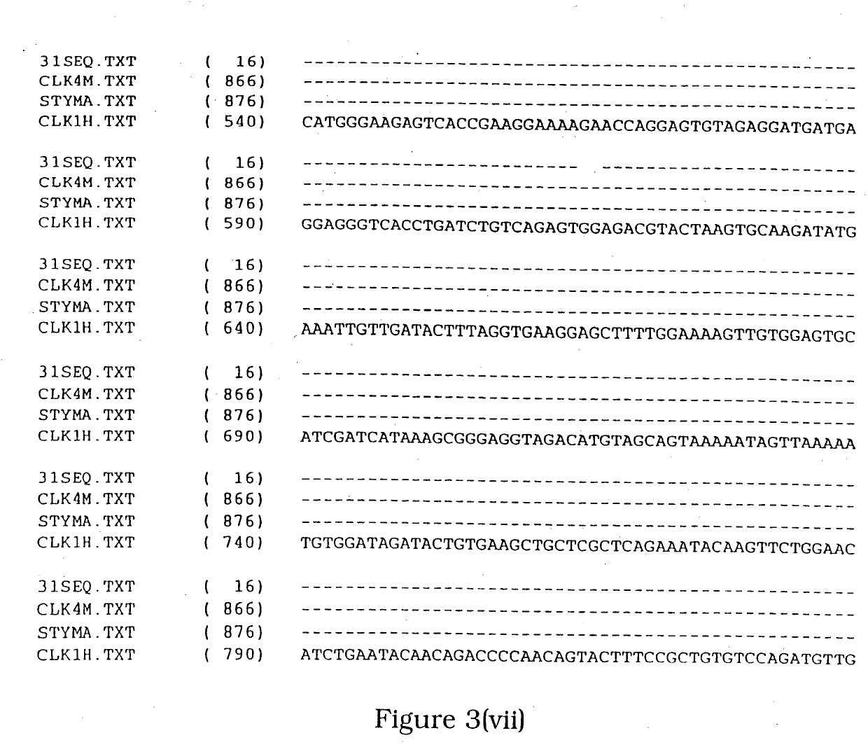

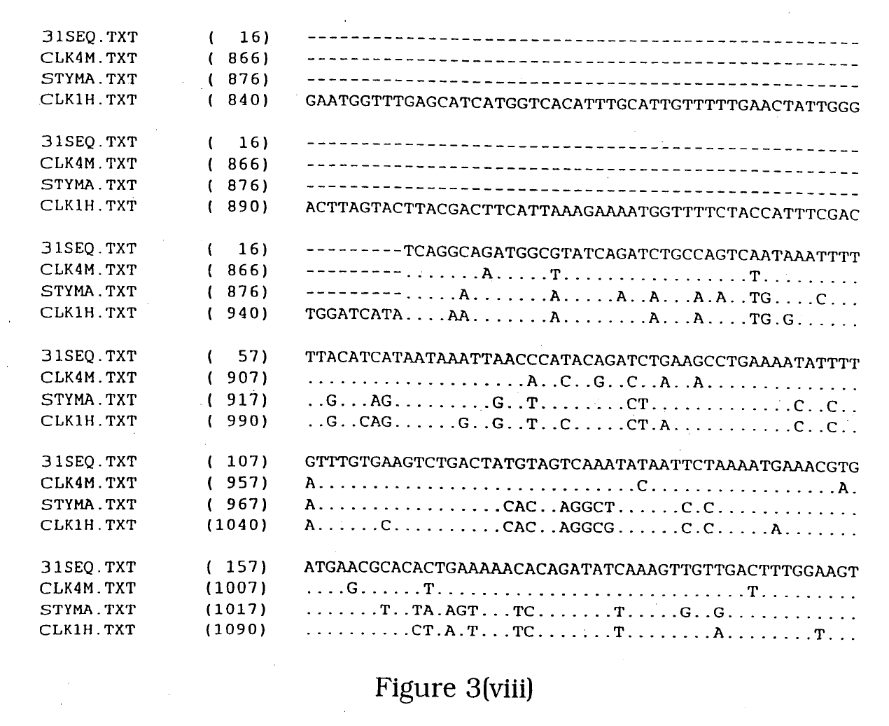

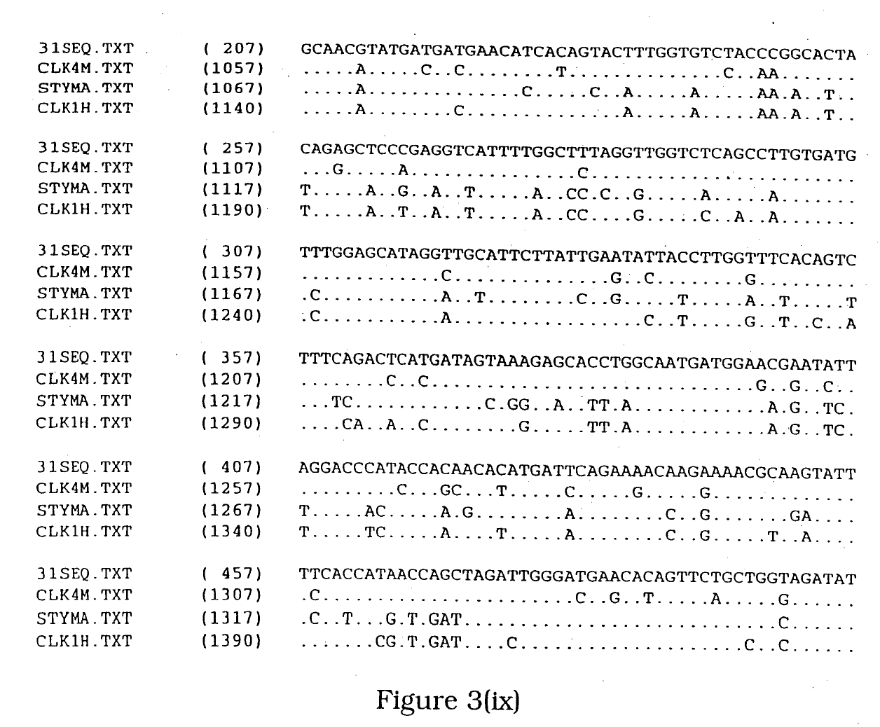

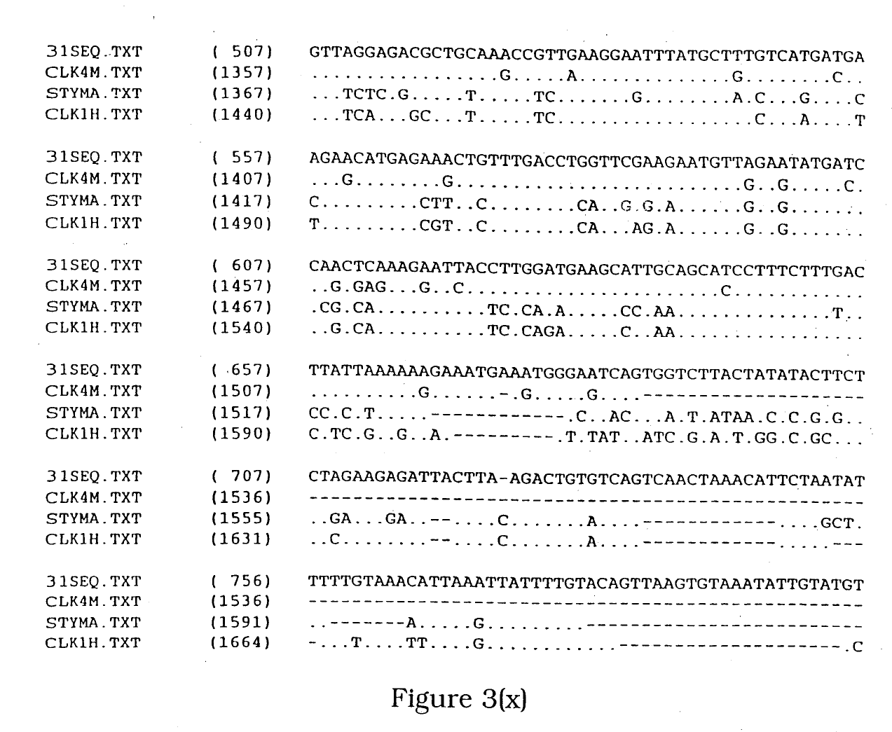

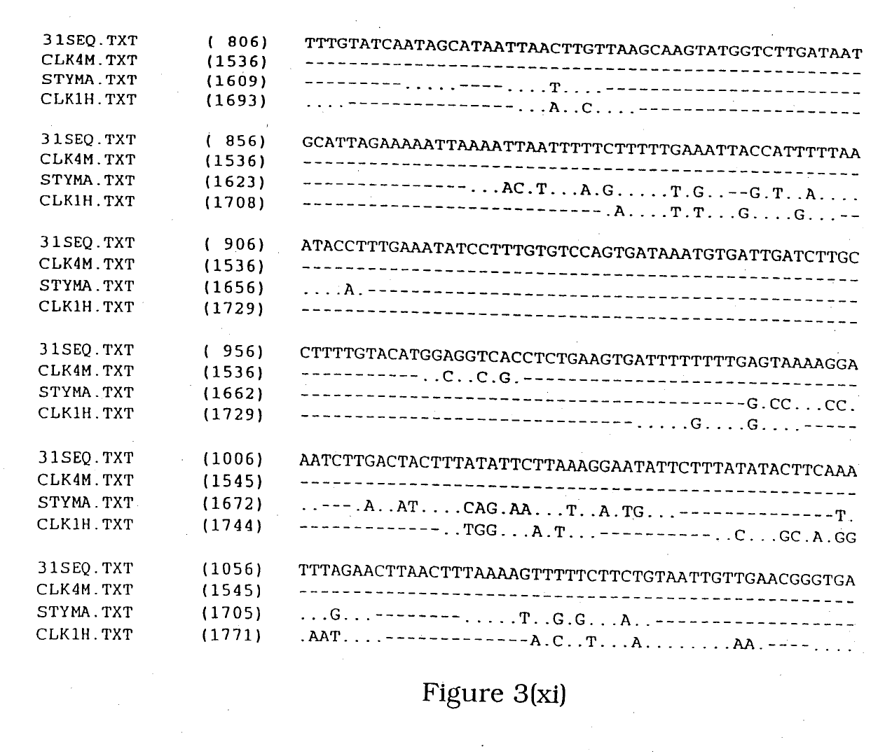

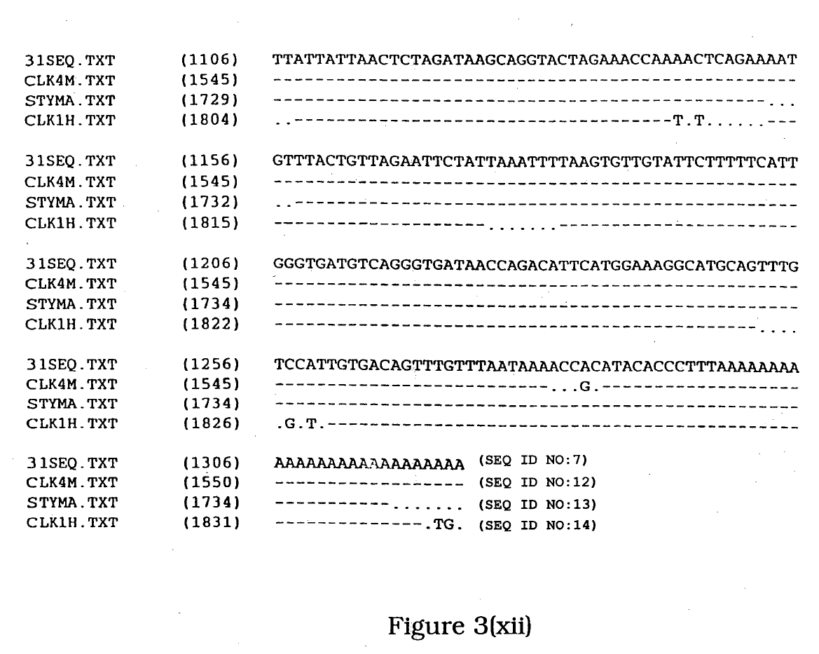

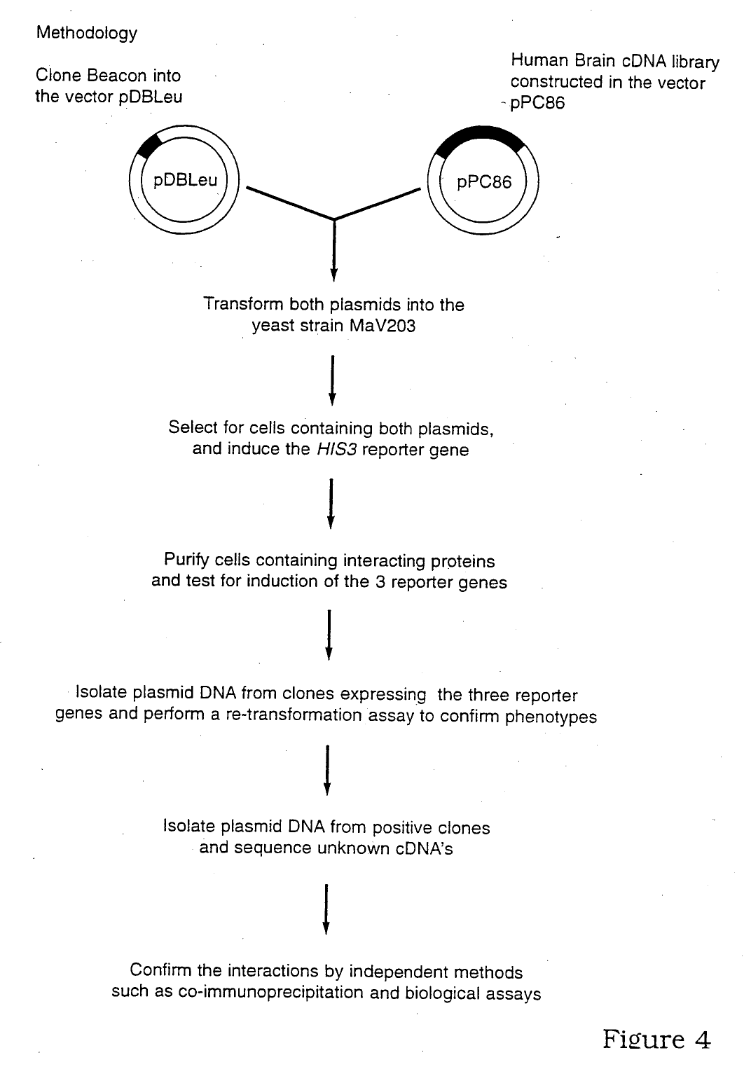

In another particularly preferred embodiment, the ligand comprises a nucleotide sequence substantially as set forth in FIG. 2 or FIG. 3 or a nucleotide sequence having at least about 50% similarity to a nucleotide sequence in FIG. 2 or FIG. 3 after optimal alignment or a nucleotide sequence capable of hybridizing to a nucleotide sequence in FIG. 2 or FIG. 3 under low stringency conditions.

For convenience, the ligand capable of interaction with beacon is referred to as “beacon ligand”. The corresponding genetic sequence encoding beacon ligand is referred to herein as “beacon ligand”. Reference herein to beacon ligand includes, where appropriate, reference to the genomic gene or cDNA as well as any naturally occurring or induced derivatives. Apart from the substitutions, deletions and/or additions to the nucleotide sequence, the present invention further encompasses mutants, fragments, parts and portions of the nucleotide sequence corresponding to the beacon ligand. The beacon gene itself may encode a “short” form or “long” form of beacon. Both the long and short forms of beacon are biologically active, and suppression of the activity of either or both forms is included in this invention. The short form of beacon can be readily synthesized in vitro, while the long form can be produced using expression vectors. A beacon ligand may be any protein including a heat shock protein and/or a cdc-like protein. In a preferred embodiment, the beacon ligand is a cdc-like kinase with strong homology to mouse clk4.

A homologue of beacon ligand or beacon ligand is considered to be a ligand from another animal species. The beacon ligand gene is exemplified herein from Psammomys obesus hypothalamus. The invention extends, however, to the homologous gene, as determined by nucleotide sequence and/or function, from humans, primates, livestock animals (e.g. cows, sheep, pigs, horses, donkeys), laboratory test animals (e.g. mice, guinea pigs, hamsters, rabbits), companion animals (e.g. cats, dogs) and captured wild animals (e.g. rodents, foxes, deer, kangaroos).

Apart from the yeast two-hybrid method, the ligand of the present invention and, in particular, beacon ligand, may also be identifiable by a number of other means. In one method, beacon or a ligand binding portion thereof is labelled with a reporter molecule and used to screen cells, cell lysate and biological fluid (including blood, serum, lymph fluid) for binding to ligand. For cloning of beacon ligand, a cDNA library is conveniently prepared and expressed in a suitable cell such as CHO cells and the presence of beacon ligand is then determined by, for example, beacon or a ligand binding portion thereof labelled with a reporter molecule.

The identification of cell types having a beacon ligand is readily determined by incubated cells with beacon, with or without neuropeptide Y (NPY) or leptin and screening for an effect. Generally, the effect is the expression of select genes or screening for signal transduction or screening for phenotypic changes. Another useful technique involves the yeast two-hybrid system. This is particularly useful where the ligand is intracellular and not expressed on the cell surface.

The present invention provides the beacon ligand gene as well as a peptide, polypeptide or protein encoded thereby. The nucleic acid molecule of this aspect of the present invention and in particular beacon ligand gene and its derivatives and homologues may be in isolated or purified form and/or may be ligated to a vector such as an expression vector. Expression may be in a eukaryotic cell line (e.g. mammalian, insect or yeast cells) or in microbial cells (e.g. E. coli) or both.

The derivatives of the beacon ligand nucleic acid molecule of the present invention include oligonucleotides, PCR primers, antisense molecules, molecules suitable for use in co-suppression and fusion nucleic acid molecules. Ribozymes and DNA enzymes are also contemplated by the present invention directed to beacon ligand or its mRNA.

Reference herein to a beacon ligand includes reference to isolated or purified naturally occurring beacon ligand molecules as well as any derivatives, homologues, analogues and mimetics thereof. Derivatives includes parts, fragments and portions of a beacon partner as well as single and multiple amino acid substitutions, deletions and/or additions to the beacon partner.

Other derivatives of a beacon ligand include chemical analogues. Analogues of a beacon ligand contemplated herein include, but are not limited to, modifications to side chains, incorporation of unnatural amino acids and/or their derivatives during peptide, polypeptide or protein synthesis and the use of crosslinkers and other methods which impose confirmational constraints on the proteinaceous molecule or their analogues.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid, contemplated herein is shown in Table 1.

TABLE 1

| | | Non-conventional | |
|---|---|---|---|
| Non-conventional amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methytryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogues by introducing covalent bonds such as forming an amide bond between the N and C termini, between two side chains or between a side chain and the N or C terminus.

All such modifications may also be useful in stabilizing the beacon partner molecule for use in in vivo administration protocols or for diagnostic purposes.

The identification of a beacon ligand permits the generation of a range of therapeutic molecules capable of modulating expression of beacon or beacon ligand or modulating the activity of beacon or beacon ligand. Modulators contemplated by the present invention includes agonists and antagonists of beacon ligand expression. Antagonists of beacon ligand expression include antisense molecules, ribozymes and co-suppression molecules. Agonists include molecules which increase promoter activity or which interfere with negative regulatory mechanisms. Antagonists of beacon ligand include antibodies and inhibitor peptide fragments. All such molecules may first need to be modified to enable such molecules to penetrate cell membranes. Alternatively, viral agents may be employed to introduce genetic elements to modulate expression of a beacon ligand. Insofar as beacon acts in association with other genes such as the ob gene which encodes leptin, the therapeutic molecules of the present invention may target both the beacon ligand and ob ligand genes or their translation products.

The present invention contemplates, therefore, a method for modulating expression of beacon ligand in a mammal, said method comprising contacting the beacon ligand gene with an effective amount of a modulator of beacon ligand expression for a time and under conditions sufficient to up-regulate or down-regulate or otherwise modulate expression of beacon ligand.

For example, a nucleic acid molecule encoding beacon ligand or a derivative or homologue thereof may be introduced into a cell to enhance the ability of that cell to produce beacon ligand. Conversely, beacon ligand antisense sequences such as oligonucleotides may be introduced to decrease the availability of beacon ligand molecules.

Another aspect of the present invention contemplates a method of modulating activity of beacon in a mammal, said method comprising administering to said mammal a modulating effective amount of a soluble beacon ligand or a derivative thereof for a time and under conditions sufficient to increase or decrease beacon activity. The derivative of beacon ligand may be a proteinaceous molecule or a chemical entity such as a product identified from a natural product library or chemical library.

One convenient means of screening for antagonists of beacon ligand when in the form of a receptor is to incubate a cell carrying a beacon ligand in the form of a receptor with beacon with or without a potential antagonist and screening for a differential effect when the antagonist is applied. Again, the effect may be gene expression, signal transduction and/or phenotypic changes.

Modulating levels of beacon ligand expression or beacon ligand activity is important in the treatment of a range of conditions such as obesity, anorexia, energy imbalance diabetes, metabolic syndrome, dyslipidemia, hypertension and insulin resistance. It may also be useful in the agricultural industry to assist in the generation of leaner animals, or where required, more obese animals. Accordingly, the mammal contemplated by the present invention includes but is not limited to humans, primates, livestock animals (e.g. pigs, sheep, cows, horses, donkeys), laboratory test animals (e.g. mice, rats, guinea pigs, hamsters, rabbits), companion animals (e.g. dogs, cats) and captured wild animals (e.g. foxes, kangaroos, deer). A particularly preferred host is a human, primate or livestock animal.

Accordingly, the present invention contemplates in one embodiment a composition comprising a soluble form of beacon ligand or a modulator of beacon ligand expression and one or more pharmaceutically acceptable carriers and/or diluents. The compositions may also comprise leptin or modulators of leptin activity or ob expression.

For brevity, all such components of such a composition are referred to as “active components”.

The compositions of active components in a form suitable for injectable use include sterile aqueous solutions (where water soluble) and sterile powders for the extemporaneous preparation of sterile injectable solutions. In all cases, the form must be sterile and must be fluid to the extent that easy syringability exists. It must be stable under the conditions of manufacture and storage and must be preserved against the contaminating action of microorganisms such as bacteria and fungi.

The carrier can be a solvent or other medium containing, for example, water, ethanol, polyol (for example, glycerol, propylene glycol and liquid polyethylene glycol, and the like), suitable mixtures thereof, and vegetable oils.

The preventions of the action of microorganisms can be brought about by various antibacterial and antifungal agents, for example, parabens, chlorobutanol, phenol, sorbic acid, thirmerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active components in the required amount in the appropriate solvent with optionally other ingredients, as required, followed by sterilization by, for example, filter sterilization, irradiation or other convenient means. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active molecules are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of-course, be varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained-release preparations and formulations.

Pharmaceutically acceptable carriers and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active component may be compounded for convenient and effective administration in sufficient amounts with a suitable pharmaceutically acceptable carrier in dosage unit form. A unit dosage form can, for example, contain the principal active component in amounts ranging from 0.5 μg to about 2000 mg. Expressed in proportions, the active compound is generally present in from about 0.5 μg to about 2000 mg/ml of carrier. In the case of compositions containing supplementary active ingredients, the dosages are determined by reference to the usual dose and manner of administration of the said ingredients.

In general terms, effective amounts of active ingredient will range from 0.01 ng/kg/body weight to above 10,000 mg/kg/body weight. Alternative amounts range from 0.1 ng/kg/body weight is above 1000 mg/kg/body weight. Active ingredients may be administered per minute, hour, day, week, month or year depending on the condition being treated. The route of administration may vary and includes intravenous, intraperitoneal, sub-cutaneous, intramuscular, intranasal, via suppository, via infusion, via drip, orally or via other convenient means. Compositions may be formulated in a variety of ways and reference may be conveniently made to Remington Pharmaceutical Sciences, 19th Edition, Mack Publishing Company, Pennsylvania, USA.

The pharmaceutical composition may also comprise genetic molecules such as a vector capable of transfecting target cells where the vector carries a nucleic acid molecule capable of modulating beacon ligand expression or beacon ligand activity. The vector may, for example, be a viral vector.

Still another aspect of the present invention is directed to antibodies to beacon ligand and its derivatives and homologues. Such antibodies may be monoclonal or polyclonal and may be selected from naturally occurring antibodies to beacon or may be specifically raised to beacon or derivatives or homologues thereof. In the case of the latter, beacon ligand or its derivatives or homologues may first need to be associated with a carrier molecule. The antibodies and/or recombinant beacon ligand or its derivatives of the present invention are particularly useful as therapeutic or diagnostic agents.

For example, beacon ligand and its derivatives can be used to screen for naturally occurring beacon or antibodies to beacon ligand which may occur in certain autoimmune diseases or where cell death is occurring. These may occur, for example in some autoimmune diseases. Alternatively, specific antibodies can be used to screen for beacon partner. Techniques for such assays are well known in the art and include, for example, sandwich assays and ELISA.

Antibodies to beacon partner of the present invention may be monoclonal or polyclonal and may be selected from naturally occurring antibodies to the beacon or may be specifically raised to the beacon or its derivatives. In the case of the latter, the beacon protein may need first to be associated with a carrier molecule. Alternatively, fragments of antibodies may be used such as Fab fragments. Furthermore, the present invention extends to recombinant and synthetic antibodies and to antibody hybrids. A “synthetic antibody” is considered herein to include fragments and hybrids of antibodies. The antibodies of this aspect of the present invention are particularly useful for immunotherapy and may also be used as a diagnostic tool or as a means for purifying beacon ligand.

For example, specific antibodies can be used to screen for beacon partner proteins. The latter would be important, for example, as a means for screening for levels of beacon partner in a cell extract or other biological fluid or purifying beacon made by recombinant means from culture supernatant fluid. Techniques for the assays contemplated herein are known in the art and include, for example, sandwich assays and ELISA.

It is within the scope of this invention to include any second antibodies (monoclonal, polyclonal or fragments of antibodies) directed to the first mentioned antibodies discussed above. Both the first and second antibodies may be used in detection assays or a first antibody may be used with a commercially available anti-immunoglobulin antibody. An antibody as contemplated herein includes any antibody specific to any region of beacon partner.

Both polyclonal and monoclonal antibodies are obtainable by immunization with the enzyme or protein and either type is utilizable for immunoassays. The methods of obtaining both types of sera are well known in the art. Polyclonal sera are less preferred but are relatively easily prepared by injection of a suitable laboratory animal with an effective amount of beacon partner or antigenic parts thereof, collecting serum from the animal, and isolating specific sera by any of the known immunoadsorbent techniques. Although antibodies produced by this method are utilizable in virtually any type of immunoassay, they are generally less favoured because of the potential heterogeneity of the product.

The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art (see, for example, Douillard and Hoffman, 1981; Kohler and Milstein, 1975, Kohler and Milstein, 1976).

Another aspect of the present invention contemplates a method for detecting beacon or a derivative or homologue thereof in a biological sample from a subject said method comprising contacting said biological sample with a beacon ligand or a derivative or homologue thereof for a time and under conditions sufficient for a complex to form, and then detecting said complex.

The presence of the complex is indicative of the presence of beacon. This assay may be quantitated or semi-quantitated to determine a propensity to develop obesity or other conditions or to monitor a therapeutic regimen.

Conveniently, beacon partner is immobilized to a solid support and a biological sample brought into contact with the immobilized molecule.

The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes to immobilize beacon partner are well-known in the art and generally consist of cross-linking, covalently binding or physically adsorbing the molecule to the solid support. An aliquot of the sample to be tested is then added to the solid phase complex and incubated for a period of time sufficient (e.g. 2-40 minutes or overnight if more convenient) and under suitable conditions (e.g. from room temperature to about 37° C.) to allow binding of beacon to is receptor. Following the incubation period, solid phase is washed and an antibody added directed to beacon. The presence of antibody binding is indicative of beacon being immobilized to its receptor.

Generally, either the beacon partner or an antibody to beacon is labelled with a receptor molecule.

In an alternative embodiment, beacon itself or a ligand binding portion thereof is labelled with a reported molecule and used to screen for beacon ligand.

By “reporter molecule” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules.

The present invention also contemplates genetic assays such as involving PCR analysis to detect beacon ligand or its derivatives.

The present invention further extends to a clk ligand which is independent of beacon or beacon-clk interaction. The clk ligand is useful for a range of applications such as acting as an antagonist for clk interaction with other ligands. The clk ligand of this aspect of the present invention, for example, is the treatment of diabetes and/or other conditions associated with clk. This aspect of the present invention further contemplates nucleic acid molecules encoding the clk ligand as well as compositions comprising the clk ligand such as pharmaceutical compositions.

The present invention is further described by reference to the following non-limiting Figures and Examples.

A summary of sequence identifiers used throughout the subject specification is provided in Table 2.

TABLE 2

| SEQUENCE | |
|---|---|
| IDENTIFIER | DESCRIPTION |
| <400>1 | Nucleotide sequence for beacon |
| <400>2 | Amino acid sequence for beacon |
| <400>3 | Complementary sequence for <400>1 |
| <400>4 | Nucleotide sequence for human beacon |
| <400>5 | Amino acid sequence for short form of beacon |
| <400>6 | Partial nucleotide sequence of pPC86 clone 31 |
| | beacon ligand |
| <400>7 | Partial nucleotide sequence of pPC86 clone 31 |
| | beacon ligand |

A summary of the single and three letter abbreviations for amino acid residues used in the present specification is provided in Table 3.

TABLE 3

| | Three-letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any residue | Xaa | X |

### EXAMPLE 1

### Animals

A Psammomys obesus colony is maintained at Deakin University, Geelong, Victoria, Australia, with the breeding pairs fed ad libitum a diet of lucerne and chow. Experimental animals were weaned at four weeks of age and given a diet of standard laboratory chow from which 12% of energy was derived from fat, 63% from carbohydrate and 25% from protein (Barastoc, Pakenham, Australia). Animals were housed individually in a temperature controlled room (22±1° C.) with a 12-12-hour light-dark cycle. The animals used in the study were aged 16-20 weeks.

### EXAMPLE 2

### Beacon Ligand in Cell Lines

This example provides means for identifying a beacon ligand (e.g. beacon receptor) in cells lines which also helps in the study of signal transduction mechanisms. Examples of suitable cell lines include 3T2, GTI-7, HepG2 and primary cultures from Israeli Sand Rats. Cells are treated with beacon with and without neuropeptide Y(NPY) or leptin and an effect observed. Generally, approximately 3-5 genes are selected for changes in expression. Analysis is generally conducted in macro-or micro-assays. Once a cell is identified carrying a beacon receptor, a cDNA library is prepared and beacon ligand identified. Signal transduction studies may also be conducted (e.g. Ca²⁺, cAMP, kinases, phosphatases).

### EXAMPLE 3

### Intracellular Beacon Ligand

In cases where a cell does not secrete beacon or synthesize it as a cellular receptor, the yeast two-hybrid system is useful for identifying beacon ligand as a binding partner of beacon.

### EXAMPLE 4

### Fusion Protein Fishing

Yeast Two-Hybrid System

(using the ProQuest Two-hybrid System available from Life Technologies)

The steps employed are as follows:
Clone the beacon gene in frame with the GAL4 activation domain of the yeast vector pPC86. Libraries available include: expression human (brain and foetal brain), mouse (embryo 8.5 or 10 days, liver, brain and lymph node), C. elegans, HeLa cell and rat (liver and brain). A custom library (Pssamomys obesus) is also created.
Transform the yeast strain MaV203 with the pDBLeu-beacon plasmid construct.
Test for self activation of the pDBLeu-beacon fusion protein and determine the concentration of 3-Amino-1,2,4-Triazole (3AT) required to titrate basal HIS3 expression levels. HIS3 encodes imidazole glycerol phosphate deydratase, an enzyme involved in histidine biosynthesis. This enzyme is specifically inhibited in a dose-dependent manner by 3AT. To maximize sensitivity of the HIS3 reporter gene, strain MaV203 expresses a basal level of HIS3. By determining the threshold of resistance to 3AT and including that concentration of 3AT in plates lacking histidine, even slight increases in HIS3 reporter is detected, enhancing the likelihood of detecting even weak protein:protein interactions.
Transform MaV203 cells containing the pDBLeu-beacon construct with pC86 library using antibiotic resistance (ampicillin and kanamycin) to select for cells that contain both plasmids and induce the HIS3 reporter gene.
Purify cells containing candidate interacting proteins then patch isolated colonies onto a masterplate.
Replica plate from the master plate onto selective plates to determine whether the three reporter genes are induced.
For cells inducing the reporter genes confirm that DB-beacon and AD-fusion protein (from the cDNA library) interact when AD-fusion protein is retested with fresh DB-beacon plasmid by either a retransformation assay or a version of plasmid shuffling.
Protein:protein interactions detected are confirmed by biological assays such as DNA sequencing of the cDNA clone to determine if the interacting protein has been previously identified. This fusion protein is also expressed in E. coli and co-precipitation experiments using antibodies raised against beacon or monoclonal antibodies raised against GAL4-AD domains (protein tags derived from the expression vectors). Other methods are employed to confirm that these proteins interact such as by surface receptor panning and fusion protein fishing.

A full length beacon sequence is cloned into the bacterial expression plasmid pGEX (Pharmacia Biotech). pGEX vectors allow for inducible, high level intracellular expression of genes as fusions with Schistoma japonicum glutathione S-transferase (GST). Induced bacterial cultures expressing pGEX-beacon are lysed by sonication in 50 mM Tris-HCl (pH 7.4) containing 1% w/v Triton X-100, 1% v/v Tween 20, 2 mM EDTA, 0.2 mM phenylmethylsulfonyl fluoride, and 10 μg/ml aprotinin. Affinity resins for the isolation of beacon binding proteins are prepared by immobilizing GST-beacon onto glutathione-Sepharose 4B beads (Pharmacia). Similar resins are prepared using GST alone to act as a control for the specificity of interaction. Affinity resins are incubated in the presence of brain lysates (or other tissue lysates of interest) and after extensive washing, proteins bound to resins are released either by bioling in SDS sample buffer or by elution with Tris-HCl (pH 7.4) containing 0.5% v/v Triton X-100. Binding proteins are then separated by SDS-PAGE and visualized by silver staining or Coomassie Blue staining. Regions of the gel containing beacon binding proteins are excized and the gel slices digested and purified by anion-exchange and reverse phase HPLC prior to amino acid sequencing.

### EXAMPLE 5

### Yeast Two-Hybrid Screening

The full length Beacon gene was cloned into the expression vector pDBLeu, which encodes the GAL 4 DNA binding domain. To screen and identify potential proteins that interact with the 73aa Beacon gene product, a commercial human brain cDNA library was purchased. This cDNA expression library was constructed in the two-hybrid activation domain vector pPC86. Following vector construction, pDBLeu—Beacon (pDB73Be) was introduced into the yeast strain MaV203 by transformation. MaV203 cells containing pDB73Be were then used to introduce, by transformation, the pPC86 cDNA library. Candidate positive clones were identified by growth on media that selects for cells containing both plasmids as well as induction of the HIS3 reporter gene. Greater than 10⁶ transformants were screened for interaction with 73aa Beacon. Of this figure, 28 clones were identified as preliminary positives as a result of induction of the HIS3 reporter gene.

Clones containing candidate interacting proteins were purified and re-tested for induction of three independent reporter genes. Of the 28 clones examined, three clones (clones 12, 16 and 31) were identified as containing potential interacting proteins due induction of all three reporter genes. To further ascertain the authenticity of interaction with 73aa Beacon, plasmid DNA from each clone was selectively isolated and re-introduced into MaV203. The re-transformation assay confirmed these clones as containing potential positive interactors with 73aa Beacon. Specifically, clones 12 and 16 were both identified as containing potential weak interacting proteins with 73aa Beacon. In contrast, clone 31 was shown to contain a strong interacting protein. The results are shown in Table 4. Plasmid DNA from each clone has been selectively isolated and partial sequences for the unknown cDNA's determined.

TABLE 4
Reporter gene expression

| Clone | −HIS | −URA | X-gal | Result |
|---|---|---|---|---|
| Clone 12 | + | + | White | +ve weak interacting proteins |
| Clone 16 | + | + | White | +ve weak interacting proteins |
| Clone 31 | +++ | +++ | Blue | +ve strong interacting proteins |

Of the three clones that exhibited positive interaction with 73aa Beacon, clones 12 and 16 revealed overlapping partial cDNA sequences that were found to be 100% homologous to that of the human heat shock protein 2 (HSPB2) in the regions examined. HSPB2 belongs to the small heat shock protein (HSP20) family and has been shown to bind and activate the myotonic dystrophy protein kinase. In vivo, heat shock proteins have been shown to interact with large numbers of different proteins, consequently interaction of HSBP2 with 73aa Beacon may prove to be biologically irrelevant in the context of Beacon action.

Partial cDNA sequence data for clone 31 (<400>7) was found to be highly homologous to that for the gene encoding mouse cdc2/CDC28-like protein kinase 4 in the region examined (Table 5). This kinase is considered to be closely related to the yeast cdc2/CDC28 kinases that have been shown to regulate the cell cycle. In a particularly preferred embodiment, the present invention is predicted on the beacon ligand being clone 31/pPC86, which has strong homology to mouse clk4.

TABLE 5
Sequence data summary

| Clone | Sequence |
|---|---|
| Clone 12/pPC86 cDNA | Partial sequence - 100% homologous to HSPB2 |
| Clone 16/pPC86 cDNA | Partial sequence - 100% homologous to HSPB2 |
| Clone 31/pPC86 cDNA | Partial sequence shows strong homology to |
| | mouse cdc2/CDC28-like protein kinase 4 |

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Barnett et al. (1994a) Diabetologia 37:671-676.
Barnett et al.(1994b) Int J Obesity 18:789-794.
Barnett et al. (1995) Diabetes Nutr Metab 8:42-47.
Bennett, S. A. and Magnus, P. (1994) Med J Aust 161:519-527.
Bouchard, C. The genetics of obesity. Boca Raton: CBC Press, 1994.
Ciechanover, A. and Schwartz, A. L. (1994) FASEB J8:182-191.
Collier et al. (1997a) Ann New York Acad Sci 827:50-63.
Collier et al. (1997b) Exp Clin Endocrinol Diabetes 105:36-37.
DeFronzo, R. A. (1988) Diabetes 37:667-688.
Douillard and Hoffman, Basic Facts about Hybridomas, in Compendium of Immunology Vol. II, ed. by Schwartz, 1981.
Kohler and Milstein, (1975) Nature 256:495-499.
Kohler and Milstein (1976) European Journal of Immunology 6:511-519.
Kopelman et al. (1994) Int J Obesity 18:188-191.
Leibowitz, S. F. (1985) Fed Proc 45:1396-1403.
National Health and Medical Research Council (1996) Acting on Australia's weight: A strategy for the prevention of overweight and obesity. Canberra: National Health and Medical Research Council.
Risk Factor Prevalence Study Management Committee. Risk Factor Prevalence Study: Survey No.3 1989.Canberra: National Heart Foundation of Australia and Australian Institute of Health, 1990.
Ravussin, E. (1995) Metabolism 44(Suppl 3):12-14.
Shafrir, E. and Gutman, A. (1993) J Basic Clin Physiol Pharm 4:83-99.
Stellar, E. (1954) Psychol Rev 61:5-22.
Walder et al. (1997a) Obesity Res 5:193-200.
Walder et al. (I997b) Int. J Obesity 22:1-7, 1998.
Waters, A-M and Bennett, S. Risk Factors for cardiovascular disease: A summary of Australian data.Canberra: Australian Institute of Health and Welfare, 1995.
Zhang et al. (1994) Nature 372:425-432.
Zimmet, P. Z. (1992) Diabetes Care 15(2):232-247.

1. A ligand of a protein or a derivative, homologue, analogue or mimetic of said protein which protein is produced in larger amounts in hypothalamus tissue of obese animals compared to lean animals.

2. A ligand according to claim 1 wherein the protein is encoded by a nucleotide sequence substantially as set forth in SEQ ID NO:1 or SEQ ID NO:4, a nucleotide sequence having at least 50% similarity after optimal alignment to SEQ ID NO:1 or SEQ ID NO:4 or a nucleotide sequence which is capable of hybridizing to SEQ ID NO:1 or SEQ ID NO:4 under low stringency conditions.

3. A ligand according to claim 1 wherein the protein comprises an amino acid sequence substantially as set forth SEQ ID NO:2 or SEQ ID NO:5 or an amino acid sequence having at least about 50% similarity after optimal alignment to SEQ ID NO:2 or SEQ ID NO:5.

4. A ligand according to claim 1 which is encoded by the nucleotide sequence substantially as set forth SEQ ID NO:6 or SEQ ID NO:7 or a nucleotide sequence capable of hybridizing to SEQ ID NO:6 or SEQ ID NO:7 under low stringency conditions.

5. A ligand according to claim 1 wherein the ligand is encoded by a nucleotide sequence substantially as set forth in SEQ ID NO:6, SEQ ID NO:7, SEQ ID NO:11, SEQ ID NO:12, SEQ ID NO:13, or SEQ ID NO:14 or a nucleotide sequence having at least about 50% similarity after optimal alignment to nucleotide sequence SEQ ID NO:6, SEQ ID NO:7, SEQ ID NO:11, SEQ ID NO:12, SEQ ID NO:13, or SEQ ID NO:14 or a nucleotide sequence capable of hybridizing to nucleotide sequence SEQ ID NO:6, SEQ ID NO:7, SEQ ID NO:11, SEQ ID NO:12, SEQ ID NO:13, or SEQ ID NO:14 under low stringency conditions.

6. A ligand according to claim 1 wherein the ligand is a heat shock protein or a cdc-like protein.

7. A ligand according to claim 6 wherein the ligand is a cdc-like kinase.

8. A method of identifying a ligand of the protein beacon or its derivatives, said method comprising introducing a first genetic construct in a yeast strain, said genetic construct comprising a nucleotide sequence encoding all or part of beacon fused to a nucleotide sequence encoding one of a DNA binding (DB) domain or an activation domain (AD) and introducing a second genetic construct into said yeast comprising a cDNA, said second genetic construct comprising elements of a cDNA library fused to a nucleotide sequence encoding the other of a DB domain or AD domain and selecting yeast cells which comprise both genetic constructs and in which a reporter gene has been subjected to two-hybrid dependent transcription.

9. A method according to claim 8 wherein the ligand is encoded by the nucleotide sequence substantially as set forth in SEQ ID NO:6 or SEQ ID NO:7 or a nucleotide sequence having at least 50% similarity after optimal alignment to SEQ ID NO:6 or SEQ ID NO:7 or a nucleotide sequence capable of hybridizing to SEQ ID NO:6 or SEQ ID NO:7 under low stringency conditions.

10. A method according to claim 8 wherein the ligand is encoded by the nucleotide sequence substantially as set forth in SEQ ID NO:6, SEQ ID NO:7, SEQ ID NO:11, SEQ ID NO:12, SEQ ID NO:13, or SEQ ID NO:14 or a nucleotide sequence having at least about 50% similarity after optimal alignment to a nucleotide sequence in SEQ ID NO:6, SEQ ID NO:7, SEQ ID NO:11, SEQ ID NO:12, SEQ ID NO:13, or SEQ ID NO:14 or a nucleotide sequence capable of hybridizing to a nucleotide sequence in SEQ ID NO:6, SEQ ID NO:7, SEQ ID NO:11, SEQ ID NO:12, SEQ ID NO:13, or SEQ ID NO:14 under low stringency conditions.

11. A ligand according to claim 8 wherein the ligand is a heat shock protein or a cdc-like protein.

12. A ligand according to claim 11 wherein the ligand is a cdc-like kinase.

13. A ligand identified by the method of claim 8.

14. A method for modulating expression of beacon ligand in a mammal, said method comprising contacting the beacon ligand gene with an effective amount of a modulator of beacon ligand expression for a time and under conditions sufficient to modulate expression of beacon ligand.

15. A method of modulating activity of beacon in a mammal, said method comprising administering to said mammal a modulating effective amount of a soluble beacon ligand or a derivative thereof for a time and under conditions sufficient to increase or decrease beacon activity.

16. A composition comprising a soluble form of beacon ligand or a modulator of beacon ligand expression and one or more pharmaceutically acceptable carriers and/or diluents.

17. A composition according to claim 16 further comprising a leptin, a modulator of leptin activity or a modulator of ob expression.
